(19) United States
(12) Patent Application Publication
(10) Pub. No.: US 2009/0018204 A1
(43) Pub. Date: Jan. 15, 2009

(54) COMPOSITION AND METHOD FOR ENHANCING HAIR GROWTH

(76) Inventor: Michael C. Brinkenhoff, Ventura, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/133,322

(22) Filed: Jun. 4, 2008

Related U.S. Application Data
(60) Provisional application No. 60/949,814, filed on Jul. 13, 2007.

(51) Int. Cl. A61K 31/164; C07C 233/00; A61Q 7/00

(52) U.S. Cl. 514/622; 564/171

(57) ABSTRACT

A composition including a molecule of the formula:

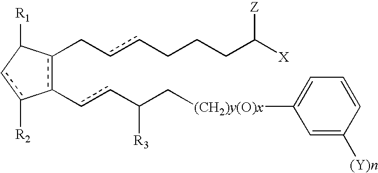

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein the alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof. A method including topically applying to a dermis or hair on the dermis an amount of a composition comprising a molecule of the formula:

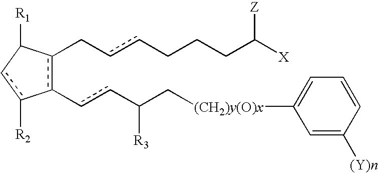

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein the alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

### CROSS-REFERENCE TO RELATED APPLICATION

This application claims the benefit of the earlier filing date of co-pending U.S. Provisional Patent Application No. 60/949,814, filed Jul. 13, 2007, and incorporated herein by reference.

### FIELD

Composition and method for stimulating the growth and coloration of human including eyelashes, including the application of a prostaglandin or prostaglandin-like compound, in the form of its solution in an ophthalmologically acceptable carrier, or incorporated into a commercial topical preparation.

### BACKGROUND

Certain therapeutic agents have been known to induce hair growth. One example is Minoxidil, 6-(1-piperidinyl)-2,4-pyrimidane-diamine 3-oxide (U.S. Pat. Nos. 3,382,247 and 3,644,363). Minoxidil was originally prepared and sold for use as an antihypertensive. It was observed that, associated with the use of Minoxidil for this latter purpose, Minoxidil use also produced an increase in hair growth and thickness as reported in U.S. Pat. Nos. 4,139,619 and 4,968,812. Today, Minoxidil is marketed under the trademark Rogaine® by Pfizer for the treatment of baldness on the scalp for men (alopecia androgenetica) and women. Another example is finasteride (Propecia®), marketed by Merck & Co. Finasteride was originally developed for benign prostatic hypertrophy, and was found to be effective in the treatment of alopecia androgenetica as reported in U.S. Pat. No. 4,968,812.

Among the drugs introduced for lowering intraocular pressure are molecules of the family prostaglandin F2. The Upjohn Company identified a prostaglandin F2α analog, commonly known as Latanoprost and whose chemical name is isopropyl-(Z)-7[(1R,2R,3R,5S)3,5-dihydroxy-2-[(3R)-3-hydroxy-5-phenylp-entyl]cyclopentyl]-5-heptenoate. Latanoprost is marketed by Pharmacia & Upjohn (currently a part of Pfizer) under the trademark Xalatan® for the reduction of elevated intraocular pressure in patients with glaucoma and ocular hypertension. The form is a Latanoprost optical solution of 0.005% (50 .mu.g/ml), and is applied by dropper directly onto the eye. One drop generally contains approximately 1.5 μg of Latanoprost. It is absorbed through the cornea where the isopropyl ester pro-drug is hydrolyzed to the acid form to become biologically active.

In the course of its use for reduction of intraocular pressure, Latanoprost has been reported to cause, in some patients, an increasing pigmentation and growth of eyelashes. U.S. Pat. No. 6,262,105 documented that the use of Latanoprost leads to increased length of lashes, increased numbers of lashes along the normal lash line, increased thickness and luster of lashes, increased auxiliary lash-like terminal hair in transitional areas adjacent to areas of normal lash growth, increased lash-like terminal hairs at the medial and lateral canthal area, increased pigmentation of the lashes, increased numbers, increased length, as well as increased luster, and thickness of fine hair on the skin of the adjacent lid, and increased perpendicular angulation of lashes and lash-like terminal hairs.

Alcon, Inc. (“Alcon”) introduced a prostaglandin F2α analog, commonly known as Travoprost whose chemical name is isopropyl (z)-7-[(1R,2R,3R,5S)-3,5-dihydroxy-2-[(1E,3R)-3-hydroxy-4-[(α,α,α-trifluoro-m-tolyl)oxyl]-1-butenyl]cyclopentyl]-5-heptenoate as a glaucoma treatment. Alcon also sought patent protection for Travoprost for growing hair in U.S. Patent Application No. 2003/0199590.

Allergan, Inc. (“Allergan”) introduced Bimatoprost whose chemical name is cyclopentane N-ethyl haptanamide-5-cis-2-(3α-hydrosy-5-phenyl-1-trans-pentenyl)-3,5-dihydroxy,[1α,2α,3α,5α) for treating glaucoma. With U.S. Patent Application No. 2003/0147823, Allergan is seeking patent protection on this molecule and similar molecules for growing hair, including eyelashes. Allergan distinguishes the Bimatoprost molecule from other prostaglandins on the basis that Bimatoprost is a prostamide.

### DETAILED DESCRIPTION

Compositions for topical application and methods of topical application of a composition to enhance hair growth are described. In one embodiment, a composition comprises an effective amount of a cyclopentane heptanoic acid, 2-cycloalkyl or arylalkyl compound represented by the following formula:

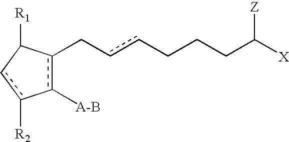

wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or alkylcarboxy groups wherein the alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

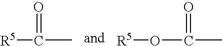

R⁵—C— and R⁵—O—C— wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above in free form or a pharmaceutically acceptable salt thereof, in association with a pharmaceutical carrier adapted for topical application to mammalian skin.

Preferably, the compound is a cyclopentane heptanoic acid, 2-(phenyl alkyl or phenyloxyalkyl) represented by the following formula:

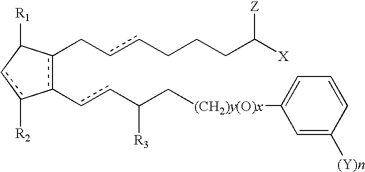

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein the alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

In another embodiment, more preferably the compound is a compound of the following formula:

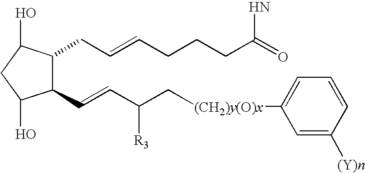

In a particular embodiment, the compound is selected from one of the following formulas:

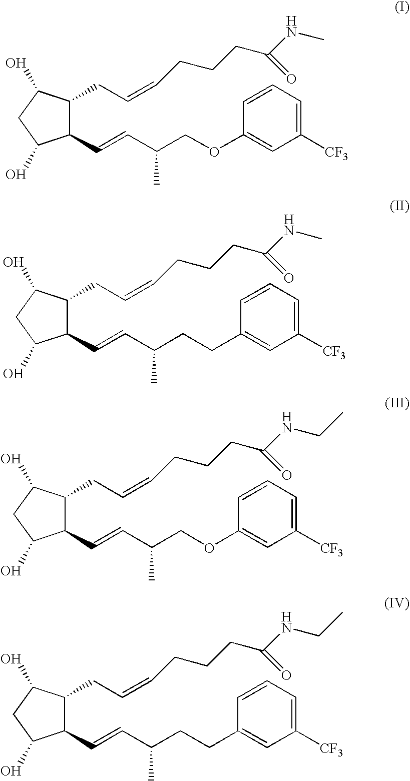

The compounds may be generally designated as prostaglandins (e.g., prostaglandin F₂_{_{α}} family) and because of the terminal amide group on their α chain may be characterized as prostamides. The compounds may be prepared using techniques known in the art including, for example, techniques described in U.S. Pat. Nos. 5,001,153, 5,422,368, 5,510,383 and 5,607,978.

In one embodiment, the one or more compounds described above is/are mixed with a dermatologically compatible vehicle or carrier. Suitable vehicles include, for example, aqueous solutions such as e.g., physiological salines, oil (e.g., castor oil), solutions or ointments. Suitable vehicles furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity.

In one embodiment, dermatological compositions for topical treatment for inducing or stimulating hair growth which comprise an effective hair growth stimulating amount of one or more compounds as defined above and a dermatologically compatible carrier are also disclosed. Effective amounts of the active compounds may be determined by one of ordinary skill in the art but will vary depending on the compound employed, frequency of application and desired result, and the compound will generally range from about 0.0000001 to about 50%, by weight, of the dermatological composition, preferably from about 0.001 to about 50%, by weight, of total dermatological composition, more preferably from about 0.1 to about 30%, by weight of the composition.

The composition may be topically applied to the dermis of, for example, a human in an area desired for hair growth or thickening of existing hair. Such location may include the scalp, eyelash area or eyelid of a male or female. Repeated application for a sustained period of time (e.g., daily for several weeks or more (e.g., one month to several months)) may be desired. In one embodiment, the composition is suitable for stimulating or inducing the growth of eyelashes. To stimulate or induce the growth of eyelashes, a composition may be applied at the base of an eyelid adjacent to or where hair follicles grow (e.g., along the lash line). Alternatively or additionally, the composition may be applied to human hair (e.g., eyelashes or eyebrows). In addition to stimulating or inducing the growth of eyelashes, it is anticipated that application to a hair follicle will increase the thickness (e.g., diameter) of the follicle.

What is claimed is:

1. A composition comprising a molecule of the formula: 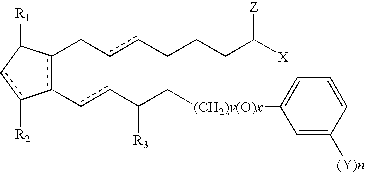
wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein the alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

2. A composition of claim 1, wherein the molecule has the formula:

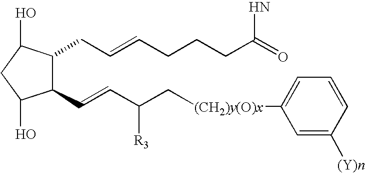

3. A composition of claim 2, wherein the molecule is of the formula:

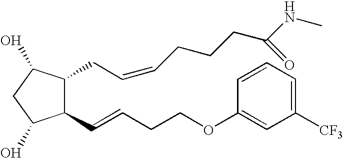

4. A composition of claim 2, wherein the molecule has the formula:

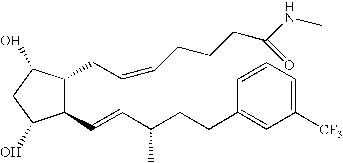

5. A composition of claim 2, wherein the molecule is of the formula:

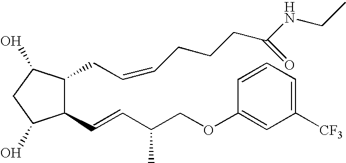

6. A composition of claim 2, wherein the molecule has the formula:

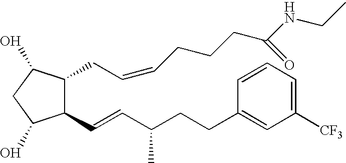

7. A method comprising:
topically applying to a dermis or hair on the dermis an amount of a composition comprising a molecule of the formula: 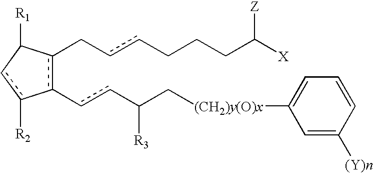
wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein the alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

8. The method of claim 7, wherein the dermis includes an eyelid.

9. The method of claim 8, wherein the molecule has the formula:

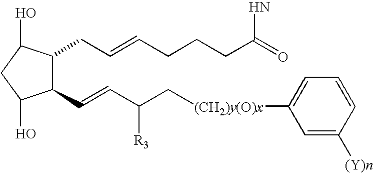

10. The method of claim 8, wherein the molecule has the formula:

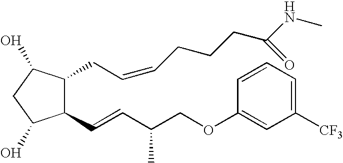

11. The method of claim 8, wherein the molecule has the formula:

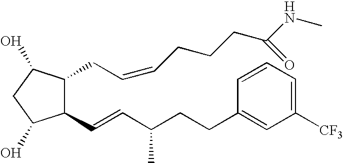

12. The method of claim 8, wherein the molecule has the formula:

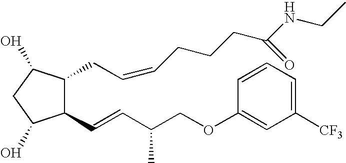

13. The method of claim 8, wherein the molecule has the formula: [chemical structure]
